(19) United States
(12) Plant Patent Application Publication
(10) Pub. No.: US 2006/0010545 P1
(43) Pub. Date: Jan. 12, 2006

(54) Osteospermum plant named 'Oste Whitey'

(50) OSTEOSPERMUM ECKLONIS; OSTEOSPERMUM

(75) Inventor: Pim Kaagman, PE Andjik (NL)

Correspondence Address: JONDLE & ASSOCIATES P.C., 858 HAPPY CANYON ROAD SUITE 230, CASTLE ROCK, CO, 80108, US

(73) Assignee: Goldsmith Seeds, Inc.

(21) Appl. No.: 10/885,543

(22) Filed: Jul. 6, 2004

(51) Int. Cl. A01H 5/00

(52) U.S. Cl. PLT/360

(57) ABSTRACT

An Osteospermum cultivar particularly distinguished by white flowers, dark green foliage and vigorous growth.

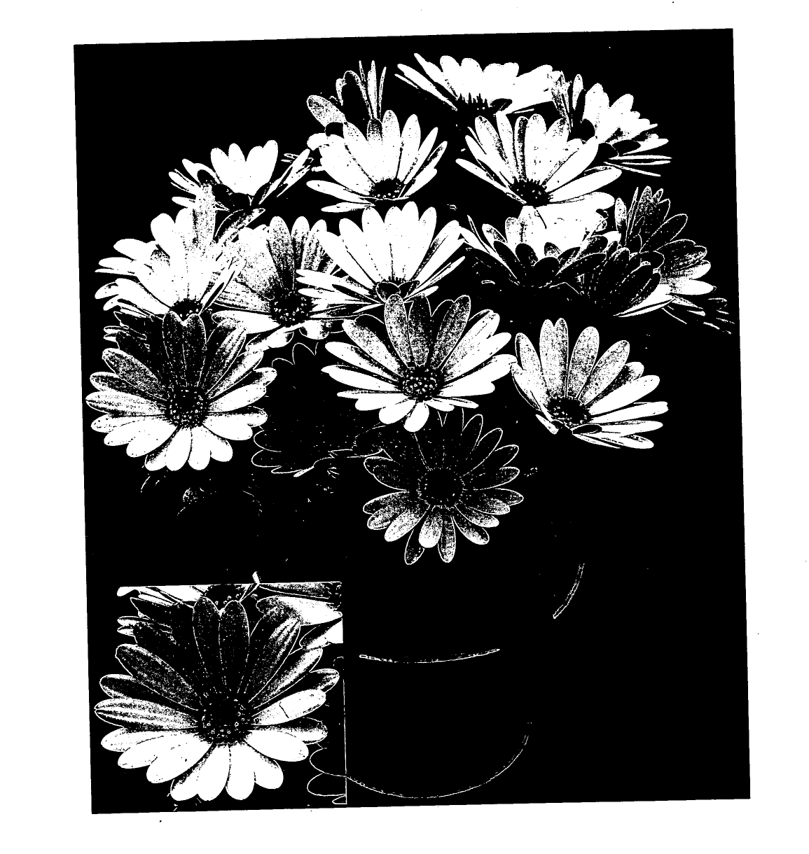

### GENUS AND SPECIES

Osteospermum ecklonis

### VARIETY DENOMINATION

‘Oste Whitey’

### BACKGROUND OF THE NEW PLANT

The variety ‘Oste Whitey’ originated from a cross made in October, 2000. The female parent is a white commercial variety ‘Starlight Neptune’ (patent status unknown). The male parent is OX-41, a very light violet, unpatented proprietary line. Seed from this cross was sown in June 2001. In October 2001, single-plant selections were made for vegetative propagation using plant habit and flower color as selection criteria. One selection became the new variety ‘Oste Whitey’.

‘Oste Whitey’ is a product of a planned breeding program intended to create new Osteospermum cultivars with white colored flowers, dark green foliage, and vigorous growth.

The new cultivar was created in 2000 in Gilroy, Calif. and has been asexually reproduced repeatedly by vegetative cuttings and tissue culture in Gilroy, Calif.; and Andijk, The Netherlands over a two ½ year period. The plant has also been trialed at Gilroy, Calif.; and Andijk, The Netherlands. The present invention has been found to retain its distinctive characteristics through successive propagation, and this novelty is firmly fixed.

### DESCRIPTION OF PHOTOGRAPH

This new Osteospermum plant is illustrated by the accompanying photograph which shows blooms, buds, and foliage of the plant in full color, the colors shown being as true as can be reasonably obtained by conventional photographic procedures. Photos were taken from plants grown in The Netherlands in the late winter/spring in a 54° greenhouse. Plants are 4½ months old, had one terminal pinch at 3 weeks old, and are grown in a 4 inch pot with 3 treatments of cyocel.

The photograph shows the mature plant with an inset photo of a close-up of the inflorescence.

### DESCRIPTION OF THE NEW CULTIVAR

The following detailed descriptions set forth the distinctive characteristics of ‘Oste Whitey’. The data, which defines these characteristics, were collected from asexual reproductions carried out in Gilroy, Calif. The plant history was taken on 5½ month old plants grown in 6 inch pots in the late winter/spring season, in a Lexan covered greenhouse under natural light and color readings were taken in the greenhouse under natural lights. Color references are primarily to the RHS Colour Chart of The Royal Horticultural Society of London (RHS) 2001. Texture description details were observed under a dissecting microscope according to The New Royal Horticultural Society Dictionary of Gardening (1992).

### THE PLANT

Classification: Osteospermum ecklonis.
Botanical: Osteospermum.
Commercial: ‘Oste Whitey’.
Form: Upright, semi-compact and mounded.
Growth and branching habit: Good vigorous, freely branched habit.
Height: From soil level to top of blooms: Approximately 40-45 cm.
Width: Approximately 35-45 cm.
Time to produce a finished flowering plant: 8-11 weeks for a 6 inch pot.
Outdoor plant performance: Herbaceous, frost tender perennial patio planters, mixed container plantings or garden beds.
Time to initiate and develop roots: Approximately 8-14 days in the greenhouse.
Root description: Fibrous, well branched, white.

### THE LEAVES

Leaf arrangement: Alternate.
Fully expanded, length: 6.5-7.0 cm; Width: 2.5-3.5 cm.
Leaf shape: Obovate.
Leaf margin: Dentate.
Apex aspect: Broadly acute.
Base aspect: Acuminate.
Leaf color: Upper surface — RHS 137B (green); Under surface RHS 137C (green).
Leaf texture: Pilose.
Venation: Pinnate.
Venation color: RHS 144C (yellow-green).
Petiole length: 0.8-0.9 cm.
Petiole diameter: 0.2 cm.
Petiole color: RHS 144C (yellow-green).
Petiole texture: Pilose.

### THE STEM

Length: 30-35 cm.
Diameter: 0.4 cm.
Stem internode length: 1.0-1.4 cm.
Color: RHS 144A (yellow-green).
Texture: Pilose.
Stem anthocyanin: None.
Peduncle color: RHS 144A (yellow-green).
Peduncle length: 9.5-13.0 cm.
Peduncle diameter: 0.2 cm.
Peduncle texture: Pilose.

### THE BUD

Shape: Ovate.
Diameter: 0.8-1.0 cm.
Length: 1.5-2.0 cm.
Color at tight bud (starting to show color): RHS 2D (yellow) with a dusting of RHS N200C (brown) in vertical lines.

### THE FLOWER

Blooming habit: Plants flower freely from the spring through fall given the proper growing conditions.
Inflorescence/floret type: Terminal and axillary inflorescences held above the foliage. Capitulum inflorescence, radially symmetrical.
Flower (limb) diameter: 7.5-8.0 cm.
Ray petal, Immature and Mature Color: Upper surface is RHS N155D (white) but whiter; 0.3-0.4 cm basal portion is RHS 70A (red-purple). Under surface is RHS N155D (white) but whiter with a suffusion of RHS 145D (yellow-green) and RHS 197D (brown) and very subdued hint of RHS 200B (brown) down the middle of the petal.
   Petal length. —3.4-3.5 cm.
   Petal width.—0.8 cm.
   Petal apex shape.—Emarginate to Praemorse.
   Petal base shape.—Cuncate.
   Petal margin.—Entire.
   Petal texture.—Papillose and hirsute on the basal margins.
   Petal quantity.—Approximately 18-20.
Disc petal, tube color: RHS 74D (red-purple).
   Shape.—Tubular, elongated
   Length.—0.4-0.5 cm.
   Diameter at apex.—0.2-0.25 cm.
   Apex shape.—5-pointed, acute.
   Apex color.—RHS 79A (purple).
   Petal quantity.—Approximately 70-80.
Phyllaries, color: RHS 138A (green).
   Length.—0.9-1.0 cm.
   Width.—0.1-0.15 cm.
   Shape.—Linear.
   Apex.—Acute.
   Texture.—Pilose and glandular hairs.
   Longevity of individual blooms.—7-14 days.
   Fragrance: None.

### THE REPRODUCTIVE ORGANS

Androecium, Pollen color: RHS 21A (yellow-orange).
   Filament color.—RHS 62D (pink).
Gynoecium, pistil: 1 per floret.
   Pistil length.—0.4 cm.
   Stigma color.—RHS 79A (purple).
   Style color.—RHS N77C (purple).
   Fruit seed set: Has not been observed.

### DISEASE AND INSECT RESISTANCE

Has not been observed.

### COMPARISON WITH KNOWN CULTIVARS

‘Oste Whitey’ differs from the female parent, ‘Starlight Neptune’ in the following ways: ‘Oste Whitey’ is earlier to flower with better flower quality and has a more compact plant habit than ‘Starlight Neptune’.

‘Oste Whitey’ differs from the male parent, OX-41 in the following ways: ‘Oste Whitey’ is later to flower, has bigger flowers, different colored flowers and the plant has more vigor than OX-41.

I claim:

1. A new and distinct cultivar of Osteospermum plant as shown and described herein.
